(19) United States
(12) Patent Application Publication
(10) Pub. No.: US 2026/0290580 A1
(43) Pub. Date: Sep. 24, 2026

(54) DISEASE DIAGNOSIS METHOD AND DEVICE USING MACHINE LEARNING-BASED LENS-FREE SHADOW IMAGING TECHNOLOGY

(71) Applicant: KOREA UNIVERSITY RESEARCH AND BUSINESS FOUNDATION, SEJONG CAMPUS, Sejong (KR)

(72) Inventors: Sungkyu SEO, Sejong (KR); Min Young BAIK, Sejong (KR); Sang Hoon SHIN, Sejong (KR)

(21) Appl. No.: 18/707,223

(22) PCT Filed: Nov. 1, 2022
(86) PCT No.: PCT/KR2022/016923

§ 371 Date: Jun. 2, 2026

(30) Foreign Application Priority Data
Nov. 5, 2021 (KR) 10-2021-0151189
Mar. 2, 2022 (KR) 10-2022-0026633

(51) Int. Cl. G16H 50/20; G06T 7/00; G06T 7/10; G16H 30/40

(52) U.S. Cl. CPC G16H 50/20; G06T 7/0012; G06T 7/10; G16H 30/40; G06T 2207/10056; G06T 2207/20081; G06T 2207/20084; G06T 2207/20112; G06T 2207/30024

(57) ABSTRACT

Various embodiments pertain to a disease diagnosis method and device using machine learning-based lens-free shadow imaging technology, which may have the configuration of acquiring a lens-free shadow image for a blood sample as a cell image, learning the cell image through a machine learning model to derive a learning result, and diagnosing a disease in relation to the blood sample on the basis of the learning result.

### CROSS-REFERENCE TO RELATED APPLICATION

This application is a National Stage application of International Patent Application No. PCT/KR2022/016923, filed on Nov. 1, 2022, which claims priority to Korean Patent Applications No. 10-2021-0151189, filed on Nov. 5, 2021, and No. 10-2022-0026633, filed on Mar. 2, 2022, each of which is hereby incorporated by reference in its entirety.

### TECHNICAL FIELD

Various embodiments pertain to a technology for diagnosing a blood-related disease, and more particularly, to a method and device for diagnosing a disease through convolution neural network (CNN)-based machine learning, from a cell image acquired through lens-free shadow imaging technology.

### BACKGROUND

Leukemia is a disease that involves the proliferation of blood cells that turn into cancer cells circulating through peripheral blood. Acute leukemia requires proper immediate treatment, and early diagnosis is very important because, without timely treatment, the disease can quickly worsen, causing death. Generally, the procedure for diagnosis of acute leukemia begins with a CBC (complete blood count) which is conducted on a patient who is experiencing sudden onset of symptoms such as feeling tired, weakness, anemia, and fever, and then, when there is any abnormality (such as the ratio between red blood cells and white blood cells) found from the CBC, peripheral blood smear examination would be conducted in which precursor cells (e.g., blasts) seen through a microscope might indicate that the patient is suspected to have leukemia. Diagnosis of leukemia can be made if there are 20% or more blasts in the peripheral blood white cells, and a bone marrow examination, flow cytometry, molecular/cellular genetic testing, etc. may be carried out to make a further diagnostic subclassification.

Observation of precursor cells through a peripheral blood smear examination is very important not only in the diagnosis of leukemia but also in tracking and monitoring post-treatment changes. Precursor cells seen in a CBC and peripheral blood smear that are performed to track and monitor changes after treatment of leukemia may indicate a sign of disease relapse. Although a more sensitive method can be used, such as detecting minimal residual disease using molecular genetic testing or flow cytometry, it does not apply to all types of leukemia and costs a lot of money and time. Peripheral blood smear examination is a method in which a drop of blood is placed and smeared on a glass slide and left to air dry, after which the slide is stained with a stain (e.g., the Wright-Giemsa stain) to be examined microscopically. It takes 1 to 2 hours to prepare a slide, and skilled labor is required to identify precursor cells through microscopy. Recently, overseas machines were introduced which automatically analyze blood smear slides by an artificial intelligence system. However, these machines are expensive and require a slide staining process as is with the existing technology.

### SUMMARY

### Technical Problem

Various embodiments provide a platform that diagnoses a disease through machine learning from a cell image that is acquired through lens-free shadow imaging technology without using a microscope, which is expensive, thus offering advantages in terms of cost and time, since even non-experts can use it.

Various embodiments provide a disease diagnosis method and device using machine learning-based lens-free shadow imaging technology.

### Technical Solution

A computer system according to various embodiments may be configured to acquire a lens-free shadow image for a blood sample as a cell image, learn the cell image through a machine learning model to derive a learning result, and diagnose a disease in relation to the blood sample on the basis of the learning result.

A method for a computer system according to various embodiments may include: acquiring a lens-free shadow image for a blood sample as a cell image; learning the cell image through a machine learning model to derive a learning result; and determining the percentage of blast cells in blood cells in the cell image on the basis of the learning result.

### Advantageous Effects

A disease diagnosis technology according to various embodiments is a technology for determining the percentage of blast cells in white blood cells by using only a few specimens, which has advantages in terms of cost because it does not use a microscope or a flow cytometer but instead uses a shadow imaging device which is much less expensive. Moreover, this technology allows for diagnosis by non-experts as opposed to the existing smear testing, by identifying cells through machine learning, thereby increasing user convenience and offering advantages in terms of cost and time. In addition, the accuracy of training datasets for machine learning can be increased by filtering cells from a shadow image according to specimen purity in a process of building up datasets for machine learning. Disease diagnosis can be done in a GPU-free, low-end computer environment because of web-based cell object detection and machine learning. Cells can be filtered by applying shadow imaging technology parameters.

### BRIEF DESCRIPTION OF THE DRAWINGS

FIG. 1 is a view schematically illustrating a computer system according to a computer system according to various embodiments.

FIG. 2 is a view schematically illustrating the lens-free shadow imaging device of FIG. 1.

FIG. 3 is a view exemplifying the lens-free shadow imaging device of FIG. 1.

FIG. 4 is a view schematically illustrating the machine learning server of FIG. 1.

FIG. 5 is a view schematically illustrating a method for a computer system according to various embodiments of the present disclosure.

FIG. 6 is a view illustrating in specific detail the step of acquiring a blood sample in FIG. 5.

FIG. 7 is a view illustrating in specific detail the step of learning the cell image in FIG. 5.

FIG. 8 is a view exemplifying the step of learning the cell image in FIG. 5.

FIG. 9 is a view illustrating a method for pre-training a machine learning model that is used in the machine learning server of FIG. 1.

FIGS. 10, 11, and 12 are views exemplifying the pre-training method of FIG. 9.

### DETAILED DESCRIPTION

Hereinafter, various embodiments of the present disclosure will be described with reference to the accompanying drawings.

FIG. 1 is a view schematically illustrating a computer system according to a computer system 100 according to various embodiments. The computer system 100 may be configured to diagnose a disease by using a machine learning-based lens-free shadow imaging technology.

Referring to FIG. 1, the computer system 100 may include at least one lens-free shadow imaging device 110 and a machine learning server 120. In this case, the lens-free shadow imaging device 110 and the machine learning server 120 may be connected via the internet 130. The lens-free shadow imaging device 110 may acquire a cell image. The machine learning server 120 may learn the cell image through a convolution neural network (CNN)-based machine learning model, for example, Alexnet. The lens-free shadow imaging device 110 may be installed at a hospital located spatially far away from the machine learning server 120 and used to diagnose patients. In certain embodiments, for a single machine learning server 120, a plurality of lens-free shadow imaging devices 110 may be respectively installed at multiple hospitals in each area.

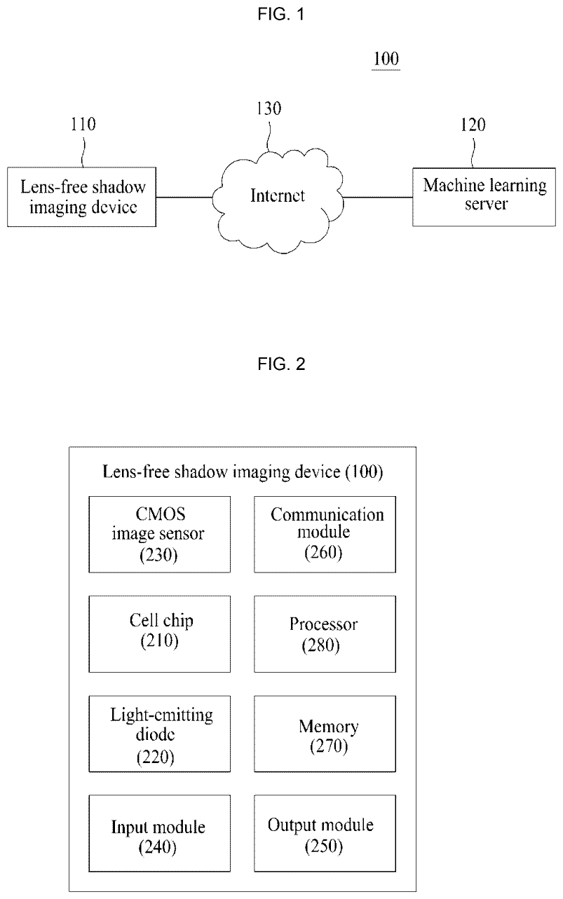

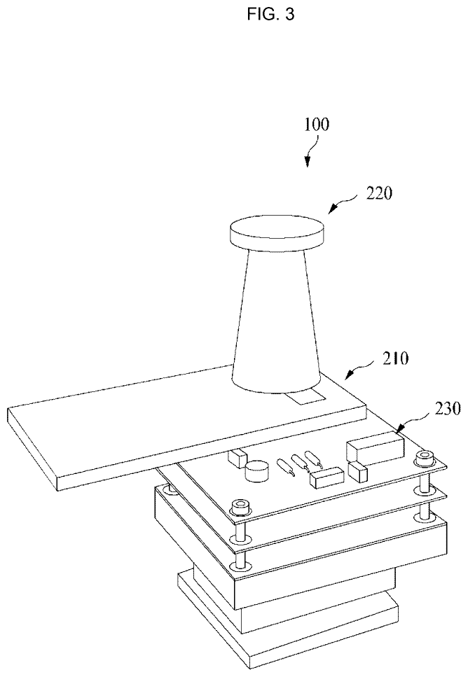

FIG. 2 is a view schematically illustrating the lens-free shadow imaging device 110 of FIG. 1. FIG. 3 is a view exemplifying the lens-free shadow imaging device 110 of FIG. 1.

Referring to FIG. 2, the lens-free shadow imaging device 110 may be configured to acquire a cell image through lens-free shadow imaging technology (LSIT). The lens-free shadow imaging device 100 may include at least one of a cell chip 210, a light-emitting diode 220, a complementary metal oxide semiconductor (CMOS) image sensor 230, an input module 240, an output module 250, a communication module 260, a memory 270, or a processor 280. In certain embodiments, at least one of the components of the lens-free shadow imaging device 110 may be omitted, and at least one another component may be added. In certain embodiments, at least two of the components of the lens-free shadow imaging device 110 may be implemented as a single integrated circuit. In this case, the lens-free shadow imaging device 110 may include at least one electronic device, and if it includes a plurality of electronic devices, the components may be configured in a distributed manner.

The cell chip 210, the light-emitting diode 220, and the CMOS image sensor 230 may acquire a lens-free shadow image for a blood sample as a cell image. In this case, the cell chip 210, the light-emitting diode 220, and the CMOS image sensor 230 may be implemented within a single device, as illustrated in FIG. 3. Unlike traditional optical microscopes which use a system of optical lenses and an expensive lamp light source, the lens-free shadow imaging device 110 may be implemented by including a lens-free CMOS image sensor 230 and a light-emitting diode 220 with a specific wavelength that has a micro pinhole mounted thereon. Thus, the lens-free shadow imaging device 110 may be implemented to achieve a wider FOV as a way of cost saving. Consequently, more cells can be observed at a time through the lens-free shadow imaging device 110 than through a microscope, since the lens-free shadow imaging device 110 has a wider FOV.

The cell chip 210 may be configured to place blood cells. In this case, the blood cells may be activated under a specific condition. Specifically, the cell chip 210 provides a space for blood cells. According to an embodiment, the cell chip 210 may include an upper substrate and a lower substrate, and the upper substrate and the lower substrate may be bonded or assembled together, with a space for blood cells in between. For example, the cell chip 210 may be made of at least one of glass, plastic, or polymer.

The light-emitting diode 220 may be configured to shed light on blood cells. Specifically, the light-emitting diode 220 may be spaced apart from the cell chip 210 and shed light on blood cells placed on the cell chip 210. In this case, the light-emitting diode 220 has a micro pinhole. The pinhole is provided to increase the coherence and illuminance of light produced by the light-emitting diode 220.

The CMOS image sensor 230 is configured to capture a lens-free shadow image of blood cells. Specifically, the CMOS image sensor 230 captures a lens-free shadow image of blood cells placed on the cell chip 210, as the light-emitting diode 220 sheds light on the cell chip 210. To this end, the CMOS image sensor 230 is placed opposite the light-emitting diode 220, with the cell chip 210 in between. According to an embodiment of the present disclosure, as illustrated in FIG. 3, the light-emitting diode 220 may be placed over the cell chip 210, and the CMOS image sensor 230 may be placed under the cell chip 210. According to another embodiment, although not shown, the light-emitting diode 220 may be placed under the cell chip 210, and the CMOS image sensor 230 may be placed over the cell chip 210.

The input module 240 may allow for input of a signal to be used for at least one component of the lens-free shadow imaging device 110. The input module 240 may include at least one of an input device configured to allow a user to enter a signal directly into the lens-free shadow imaging device 110 or a sensor device configured to generate a signal by sensing a change in surroundings. For example, the input device may include at least one of a microphone, a mouse, or a keyboard. In certain embodiments, the input device may include at least one of touch circuitry configured to sense a touch or sensor circuitry configured to measure the strength of a force generated by a touch.

The output module 250 may output information to the outside of the lens-free shadow imaging device 110. The output module 250 may include at least one of a display device configured to visually output information or an audio output device capable of outputting information as an audio signal. For example, the display device may include at least one of a display, a holographic device, or a projector. In certain embodiments, the display device may be implemented as a touchscreen, assembled together with at least one of the touch circuitry or sensor circuitry of the input module 110. For example, the audio output device may include at least one of a speaker or a receiver.

The communication module 260 may perform communication with a device external to the lens-free shadow imaging device 110. The communication module 260 may establish a communication channel between the lens-free shadow imaging device 110 and the external device and perform communication with the external device over the communication channel. Here, the external device may include at least one of a satellite, a base station, a server, or another electronic device. The communication module 260 may include at least one of a wired communication module or a wireless communication module. The wired communication module may be connected with wires to the external device and communicate in a wired manner. The wireless communication module may include at least one of a short-range communication module or a long-range communication module. The short-range communication module may communicate with the external device through short-range communication. For example, the short-range communication may include at least one of Bluetooth, WiFi direct, or infrared data association (IrDA). The long-range communication module may communicate with the external device through long-range communication. Here, the long-range communication module may communicate with the external device via a network. For example, the network may include at least one of a cellular network, the Internet, or a computer network such as LAN (local area network) or WAN (wide area network).

The memory 270 may store various data that is used by at least one component of the lens-free shadow imaging device 110. For example, the memory 270 may include at least of one of a volatile memory or a nonvolatile memory. The data may include at least one program and input data or output data related thereto. The program may be stored in the memory 270 as software including at least one instruction, and, for example, may include at least one of an operating system (OS), middleware, and an application.

The processor 280 may control at least one component of the lens-free shadow imaging device 110 by executing the program of the memory 270. Thus, the processor 280 may perform data processing or operation. Here, the processor 280 may execute an instruction stored in the memory 270. The processor 280 may detect information about a patient that is inputted through the input module 240. Also, the processor 280 may transmit a cell image of the patient to the machine learning server 120 through the communication module 260. Moreover, the processor 280 may receive a learning result for the cell image through the communication module 260. Thus, the processor 280 may output a disease diagnosis result based on the learning result through the output module 250.

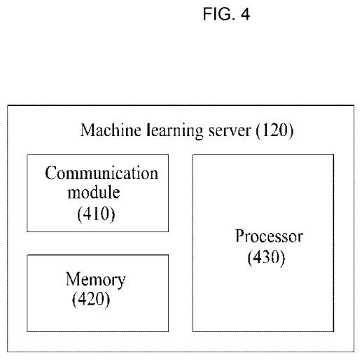

FIG. 4 is a view schematically illustrating the machine learning server 120 of FIG. 1.

Referring to FIG. 4, the machine learning server 120 may be configured to perform machine learning on a cell image. The machine learning server 120 may include at least one of a communication module 410, a memory 420, or a processor 430. In certain embodiments, at least one of the components of the machine learning server 120 may be omitted, and at least one another component may be added. In certain embodiments, at least two of the components of the machine learning server 120 may be implemented as a single integrated circuit. In this case, the machine learning server 120 may include at least one server, and if it includes a plurality of servers, the components may be configured in a distributed manner.

The communication module 410 may perform communication with a device external to the machine learning server 120. The communication module 410 may establish a communication channel between the machine learning server 120 and the external device and perform communication with the external device over the communication channel. Here, the external device may include at least one of an electronic device, a satellite, a base station, or another server. The communication module 410 may include at least one of a wired communication module or a wireless communication module. The wired communication module may be connected with wires to the external device and communicate in a wired manner. The wireless communication module may include at least one of a short-range communication module or a long-range communication module. The short-range communication module may communicate with the external device through short-range communication. For example, the short-range communication may include at least one of Bluetooth, WiFi direct, or infrared data association (IrDA). The long-range communication module may communicate with the external device through long-range communication. Here, the long-range communication module may communicate with the external device via a network. For example, the network may include at least one of a cellular network, the Internet, or a computer network such as LAN (local area network) or WAN (wide area network).

The memory 420 may store various data that is used by at least one component of the machine learning server 120. For example, the memory 420 may include at least of one of a volatile memory or a nonvolatile memory. The data may include at least one program and input data or output data related thereto. The program may be stored in the memory 420 as software including at least one instruction, and, for example, may include at least one of an operating system (OS), middleware, and an application.

The processor 430 may control at least one component of the machine learning server 120 by executing the program of the memory 420. Thus, the processor 430 may perform data processing or operation. Here, the processor 430 may execute an instruction stored in the memory 420. The processor 430 may receive a cell image from the lens-free shadow imaging device 110 through the communication module 410. Also, the processor 430 may learn the cell image through a machine learning model. In this case, the machine learning module may include a convolution neutral network (CNN)-based machine learning module, for example, Alexnet. Thus, the processor 430 may transmit a learning result to the lens-free shadow imaging device 110 through the communication module 410.

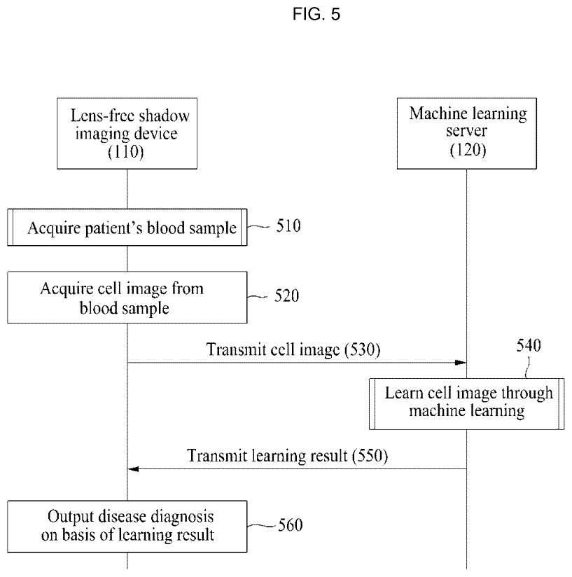

FIG. 5 is a view schematically illustrating a method for a computer system 100 according to various embodiments of the present disclosure. The method for the computer system 100 may be configured to diagnose a disease by using a machine learning-based lens-free shadow imaging technology.

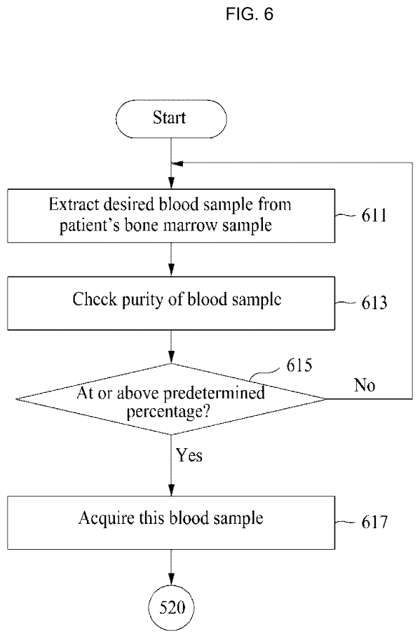

Referring to FIG. 5, in the step S510, the lens-free shadow imaging device 110 may acquire a patient's blood sample. In this case, the blood sample may have a plurality of blood cells. According to an embodiment, the lens-free shadow imaging device 110 may acquire a patient's blood sample by taking the patient's blood sample in vitro and then placing it on the cell chip 210. According to another embodiment, the lens-free shadow imaging device 110 may acquire a patient's blood sample by extracting the patient's blood sample from a bone marrow sample taken from the patient and then placing it on the cell chip 210. This will be described in more detail with reference to FIG. 6. In certain embodiments, the processor 280 may detect information about the patient which is inputted through the input module 240.

FIG. 6 is a view illustrating in specific detail the step 510 of acquiring a blood sample in FIG. 5.

Referring to FIG. 6, in the step 611, a desired blood sample may be extracted from a patient's bone marrow sample. To this end, a bone marrow sample may be taken from a patient in a hospital or the like. Afterwards, a blood sample, e.g., CD3+, and the rest of the sample, may be separated from a bone marrow sample through flow cytometry such as MACS (magnetic-activated cell sorting), and thereby only the blood sample may be extracted. In the step 613, the purity of the blood sample may be checked. In this case, the purity of the blood sample may be checked through flow cytometry such as MACS (magnetic-activated cell sorting). Afterwards, in the step 615, it may be determined whether the purity of the blood sample is at or above a predetermined percentage. For example, the predetermined percentage may be 70%.

In the step 615, if it is determined that the purity of the blood sample is below the predetermined percentage, the process may return to the step 611. In this case, a desired blood sample may be re-extracted from another bone marrow sample of the patient. To this end, another bone marrow sample may be taken from the patient in the hospital.

Meanwhile, in the step 615, if it is determined that the purity of the blood sample is at or above a predetermined percentage, this blood sample may be acquired in the step 617. Afterwards, the process may return to FIG. 5 to carry out the step 520.

Referring again to FIG. 5, the lens-free shadow imaging device 110 may acquire a cell image for blood cells from the blood sample in the step 520. Specifically, the blood sample may be placed on the cell chip 210, and then, as the light-emitting diode 220 sheds light on the blood sample, the CMOS image sensor 230 may capture a lens-free shadow image for the blood cells from the blood sample. Thus, the processor 280 may acquire the lens-free shadow image as a cell image.

Subsequently, the lens-free shadow imaging device 110 may transmit the cell image to the machine learning server 120 in the step 530. Specifically, the processor 280 may upload the patient's cell image onto the machine learning server 120 through the communication module 260. In certain embodiments, the processor 280 may transmit to the machine learning server 120 at least some of the information about the patient, along with the patient's cell image. Thus, the machine learning server 120 may receive the cell image from the lens-free shadow imaging device 110 in the step 530. Specifically, the processor 430 may receive the cell image from the lens-free shadow imaging device 110 through the communication module 410.

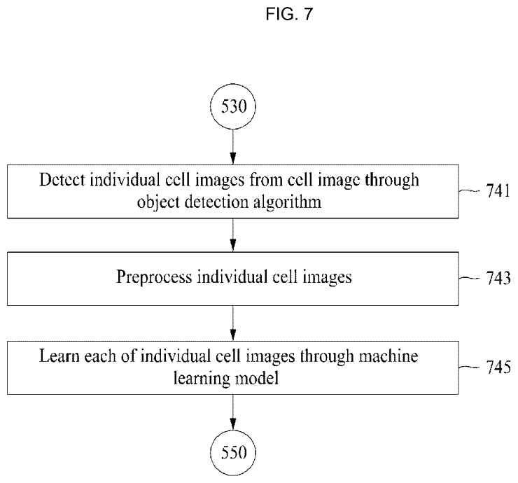

Then, the machine learning server 120 may learn the cell image through a machine learning model in the step 540. In this case, the machine learning model may include a convolution neural network (CNN)-based machine learning model, for example, Alexnet. This will be described in more detail with reference to FIG. 7.

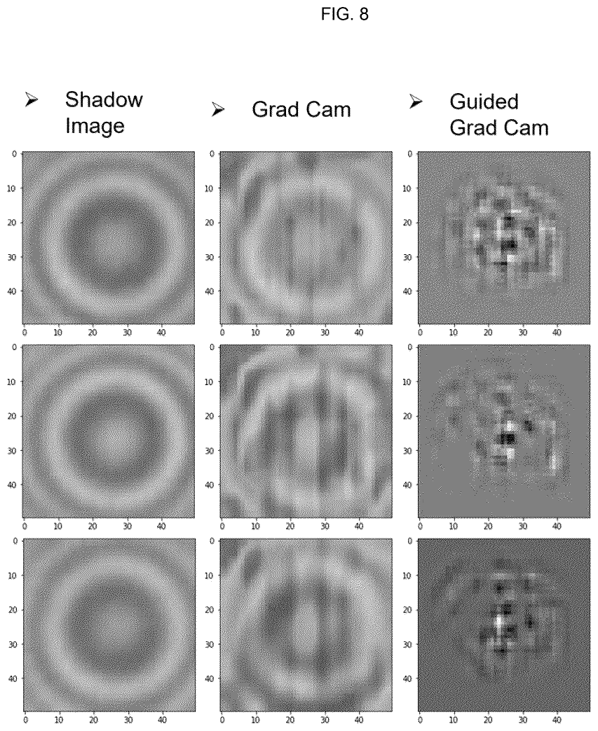

FIG. 7 is a view illustrating in specific detail the step 540 of learning the cell image in FIG. 5. FIG. 8 is a view exemplifying the step 540 of learning the cell image in FIG. 5.

Referring to FIG. 7, the machine learning server 120 may perform automatic detection on the cell image through an object detection algorithm in the step 741. In this case, the processor 430 may detect a plurality of individual cell images from the cell image. In this instance, each of the individual cell images may be an image of each blood cell in the cell image. For example, the processor 430 may cut the cell image and detect individual cell images.

Next, the machine learning server 120 may perform preprocessing on each of the individual cell images in the step 743. In this case, the processor 430 may perform preprocessing on each of the individual cell images based on the purity of each blood cell. Here, the processor 430 may remove debris or noise from each of the individual cell images. Thus, at least one individual cell image that is not correct may be removed from the individual cell images.

Next, the machine learning server 120 may perform learning on each of the individual cell images through a machine learning model in the step 745. In this case, the machine learning model may include a convolution neural network (CNN)-based machine learning model, for example, Alexnet. Here, the machine learning model may be pre-trained. Thus, the processor 430 may detect blast cells from the blood cells in the individual cell images. Also, the machine learning server 120 may acquire a learning result. In this case, the learning result may include the percentage of blast cells in the blood cells in the cell image. For example, the processor 430 may visualize differences between the individual cell images with respect to predetermined characteristics as illustrated in FIG. 8, by learning each of the individual cell images based on the predetermined characteristics. Afterwards, the process may return to FIG. 5 to carry out the step 550.

Referring again to FIG. 5, the machine learning server 120 may transmit the learning result to the lens-free shadow imaging device 110 in the step 550. Specifically, the processor 430 may transmit the learning result to the lens-free shadow imaging device 110 through the communication module 410. Thus, the lens-free shadow imaging device 1110 may receive the learning result from the machine learning server 120 in the step 747. Specifically, the processor 280 may receive the learning result for the cell image through the communication module 260.

Lastly, the lens-free shadow imaging device 110 may output a disease diagnosis result based on the learning result in the step 550. Specifically, the processor 280 may output a disease diagnosis result based on the learning result through the output module 250. In this case, the processor 280 may check the percentage of blast cells in blood cells in the cell image and therefore derive a disease diagnosis result. Here, if the percentage of blast cells in the blood cells in the cell image is at or above a predetermined percentage, the processor 280 may determine the presence of a disease.

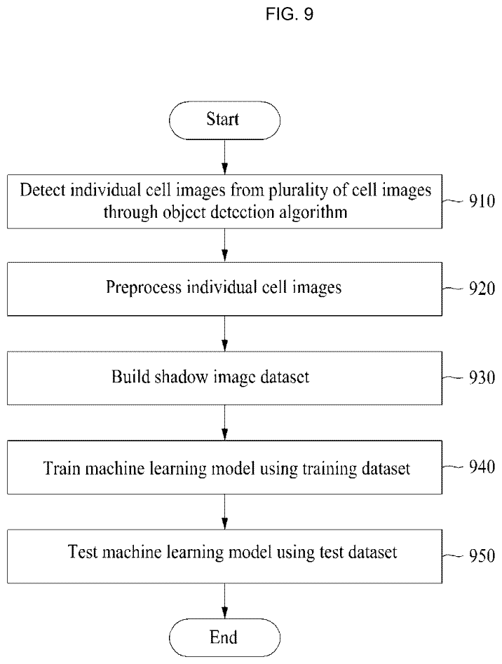

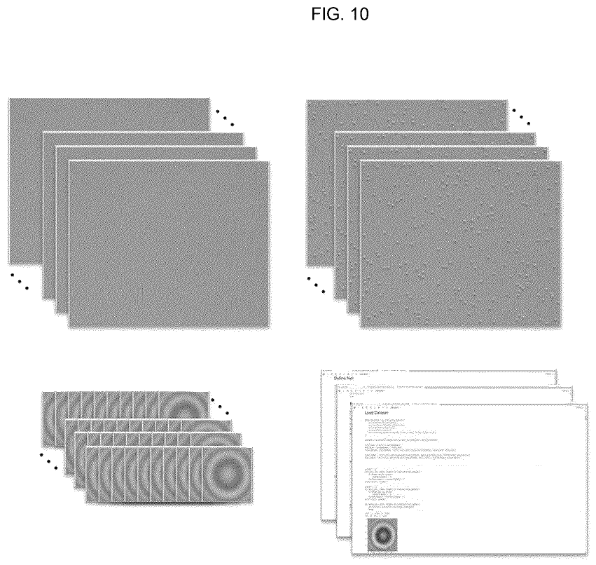

FIG. 9 is a view illustrating a method for pre-training a machine learning model that is used in the machine learning server 120 of FIG. 1. FIGS. 10, 11, and 12 are views exemplifying the pre-training method of FIG. 9.

Referring to FIG. 9, the machine learning server 120 may perform automatic detection on a plurality of cell images through an object detection algorithm in the step 910. In this case, the processor 430 may detect a plurality of individual cell images from each cell image, as illustrated in FIG. 10. In this instance, each of the individual cell images may be an image of each blood cell in the cell image. For example, the processor 430 may cut each cell image and detect individual cell images.

Next, the machine learning server 120 may perform preprocessing on each of the individual cell images of the plurality of cell images in the step 920. In this case, the processor 430 may perform preprocessing on each of the individual cell images based on the purity of each blood cell. Here, the processor 430 may remove debris or noise from each of the individual cell images. Thus, at least one individual cell image that is not correct may be removed from the individual cell images.

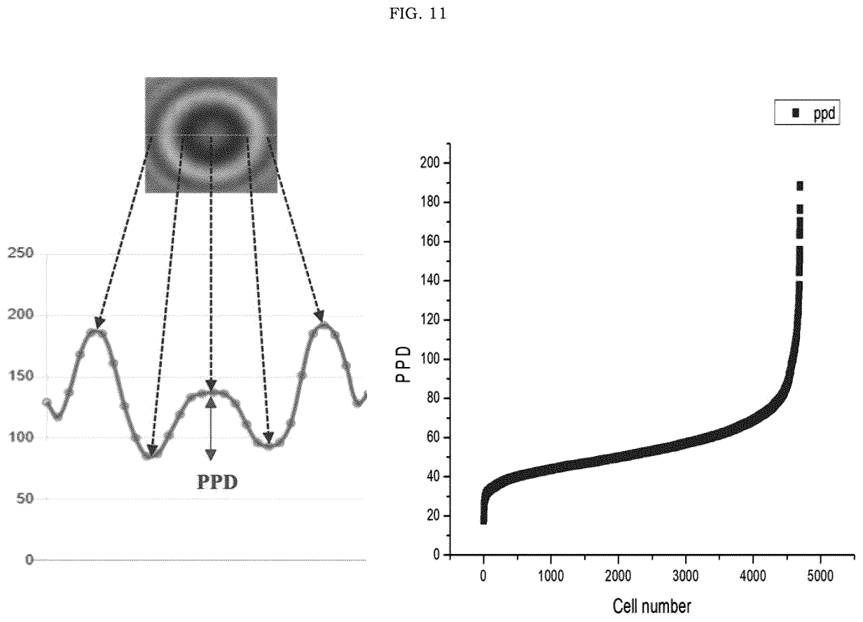

Next, the machine learning server 120 may build a shadow image dataset in the step 930. In this case, when a PPD (peak to peak distance) parameter is defined as illustrated in FIG. 11, the processor 430 may build a dataset based on PPD values of individual cell images as illustrated in FIG. 11. Here, the dataset may be built solely from individual cell images having PPD values corresponding to a range of 40 to 60, among 5,000 individual cell images. Such a dataset may be classified into a training dataset and a test dataset.

Next, the machine learning server 120 may train a machine learning model by using a dataset in the step 940. In this case, the machine learning model may include a convolution neural network (CNN)-based machine learning model, for example, Alexnet. Specifically, the processor 430 may train the machine learning model by using a training dataset. Afterwards, the machine learning server 120 may test the machine learning model by using a dataset in the step 950. Specifically, the processor 430 may test the machine learning model by using a test dataset.

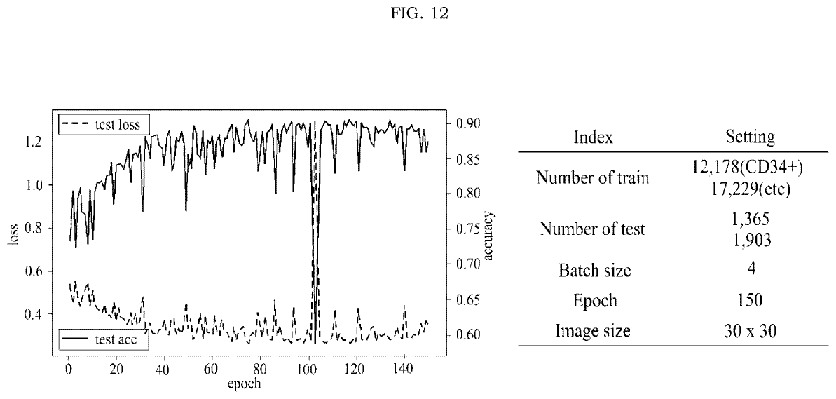

To verify this, as illustrated in FIG. 12, a test was conducted. In this case, individual cell images used in the training include 12,178 individual cell images in relation to CD34+ and 17,229 individual cell images in relation to the remaining cells separated from CD34+ through MACS. Meanwhile, the individual cell images used in the test include 1,365 individual cell images in relation to CD34+ and 1,903 individual cell images in relation to the remaining cells separated from CD34+ through MACS. The batch size used for the training was 4, the image size was 30×30, and the number of epochs was 150. As a consequence, the trained machine learning model showed about 90% correct answer rates and about 26% loss.

The present disclosure relates to a platform capable of diagnosing a disease by combining lens-free shadow imaging technology with machine learning, and uses lens-free shadow imaging technology and a CNN-based machine learning platform of Alexnet.

A disease diagnosis platform proposed in the present disclosure is a platform that is applied not only to leukemia but also to all kinds of blood-related diseases, which diagnoses a disease by learning cell forms found in the disease to be identified.

A disease diagnosis platform proposed in the present disclosure may be applied to develop a variety of models, such as a business model in which the platform is used at hospitals to diagnose a patient suspected to have a disease with mild symptoms as a pre-emptive examination method of disease diagnosis that is simpler than blood smear examination, or a business model that allows consumers to the platform themselves right on the spot.

Currently, various biological analysis methods have a competitive edge in the marketplace, and, in addition, they have enough potential to make inroads into developing nations where disease diagnosis is not very affordable, since they allow examination by cheap and unskilled labor through the low-cost, lens-free shadow imaging technology and the efficient machine learning.

A disease diagnosis platform proposed in the present disclosure is applicable in a variety of enterprises that are interested in novel examination methods of disease diagnosis, and also can be utilized as a simple method for on-site diagnosis of diseases that is conducted at domestic and foreign hospitals before blood smear examination.

To sum up, a computer system 100 according to various embodiments may be configured to acquire a lens-free shadow image for a blood sample as a cell image, learn the cell image through a machine learning model to derive a learning result, and diagnose a disease in relation to the blood sample on the basis of the learning result.

According to various embodiments, the computer system 100 may include a lens-free shadow imaging device 110 that is configured to acquire a cell image.

According to various embodiments, the lens-free shadow imaging device 110 may include a cell chip 210 where the blood sample is placed, a light-emitting diode 220 configured to shed light on the blood sample, and a CMOS image sensor 230 configured to capture a lens-free shadow image of the blood sample.

According to various embodiments, the computer system 100 may further include a machine learning server 120 having the machine learning model.

According to various embodiments, the machine learning server 120 may be configured to receive the cell image from the lens-free shadow imaging device 110 and learn the cell image through the machine learning model to derive a learning result.

According to various embodiments of the present disclosure, the blood sample may have a plurality of blood cells, and the machine learning server 120 may be configured to cut the cell image and detect a plurality of individual cell images for each of the blood cells through an object detection algorithm and to learn the individual cell images and detect blast cells from the blood cells.

According to various embodiments, the learning result may include the percentage of blast cells in the blood cells in the cell image.

According to various embodiments, the lens-free shadow imaging device 110 may be configured to receive the learning result from the machine learning server 120 and diagnose a disease in relation to the blood sample on the basis of the learning result.

According to various embodiments, the computer system 100 may be configured to diagnose a disease if the percentage of blast cells in the blood cells in the cell image is at or above a predetermined percentage.

According to various embodiments, the blood cells may be CD4+ cells.

According to various embodiments, the machine learning model may include Alexnet which is based on a convolution neural network.

Meanwhile, a method for the computer system 100 according to various embodiments may include the step 520 of acquiring a lens-free shadow image for a blood sample as a cell image, the step 540 of learning the cell image through a machine learning model to derive a learning result; and the step 560 of determining the percentage of blast cells in blood cells in the cell image on the basis of the learning result.

According to various embodiments, the step 520 of acquiring a lens-free shadow image as a cell image may be performed by a lens-free shadow imaging device 110.

According to various embodiments, the step 540 of learning the cell image to derive the learning result may be performed by a machine learning server 120 having the machine learning model.

According to various embodiments, the blood sample may have a plurality of blood cells, and the step 540 of learning the cell image to derive the learning result may include the step 741 of cutting the cell image and detecting a plurality of individual cell images for each of the blood cells through an object detection algorithm and the step 745 of learning the individual cell images and detecting blast cells from the blood cells.

According to various embodiments, the step 560 of determining the percentage of blast cells in the blood cells in the cell image may be performed by the lens-free shadow imaging device 110.

The above-described method may be provided as a computer program stored in a computer-readable recording medium for execution on a computer. The medium may be a type of medium that continuously stores a program executable by a computer, or temporarily stores the program for execution or download. In addition, the medium may be a variety of recording means or storage means having a single piece of hardware or a combination of several pieces of hardware, and is not limited to a medium that is directly connected to any computer system, and accordingly, may be present on a network in a distributed manner. An example of the medium includes a medium configured to store program instructions, including a magnetic medium such as a hard disk, a floppy disk, and a magnetic tape, an optical medium such as a CD-ROM and a DVD, a magnetic-optical medium such as a floptical disk, and a ROM, a RAM, a flash memory, and so on. In addition, other examples of the medium may include an app store that distributes applications, a site that supplies or distributes various software, and a recording medium or a storage medium managed by a server.

The methods, operations, or techniques of this disclosure may be implemented by various means. For example, these techniques may be implemented in hardware, firmware, software, or a combination thereof. Those skilled in the art will further appreciate that various illustrative logical blocks, modules, circuits, and algorithm steps described in connection with the disclosure herein may be implemented in electronic hardware, computer software, or combinations of both. To clearly illustrate this interchangeability of hardware and software, various illustrative components, blocks, modules, circuits, and steps have been described above generally in terms of their functionality. Whether such a function is implemented as hardware or software varies depending on design requirements imposed on the particular application and the overall system. Those skilled in the art may implement the described functions in varying ways for each particular application, but such implementation should not be interpreted as causing a departure from the scope of the present disclosure.

In a hardware implementation, processing units used to perform the techniques may be implemented in one or more ASICs, DSPs, digital signal processing devices (DSPDs), programmable logic devices (PLDs), field programmable gate arrays (FPGAs), processors, controllers, microcontrollers, microprocessors, electronic devices, other electronic units designed to perform the functions described in the present disclosure, computer, or a combination thereof.

Accordingly, various example logic blocks, modules, and circuits described in connection with the present disclosure may be implemented or performed with general purpose processors, DSPs, ASICs, FPGAs or other programmable logic devices, discrete gate or transistor logic, discrete hardware components, or any combination of those designed to perform the functions described herein. The general purpose processor may be a microprocessor, but in the alternative, the processor may be any related processor, controller, microcontroller, or state machine. The processor may also be implemented as a combination of computing devices, for example, a DSP and microprocessor, a plurality of microprocessors, one or more microprocessors associated with a DSP core, or any other combination of the configurations.

In the implementation using firmware and/or software, the techniques may be implemented with instructions stored on a computer-readable medium, such as random access memory (RAM), read-only memory (ROM), non-volatile random access memory (NVRAM), programmable read-only memory (PROM), erasable programmable read-only memory (EPROM), electrically erasable PROM (EEPROM), flash memory, compact disc (CD), magnetic or optical data storage devices, etc. The instructions may be executable by one or more processors, and may cause the processor(s) to perform certain aspects of the functions described in the present disclosure.

Although the embodiments described above have been described as utilizing aspects of the currently disclosed subject matter in one or more standalone computer systems, embodiments are not limited thereto, and may be implemented in conjunction with any computing environment, such as a network or distributed computing environment. Furthermore, aspects of the subject matter in the present disclosure may be implemented in multiple processing chips or devices, and storage may be similarly influenced across a plurality of devices. Such devices may include PCs, network servers, and portable devices.

Although the present disclosure has been described in connection with some examples herein, various modifications and changes can be made without departing from the scope of the present disclosure, which can be understood by those skilled in the art to which the present disclosure pertains. In addition, such modifications and changes should be considered within the scope of the claims appended herein.

1. A computer system, which is configured to acquire a lens-free shadow image for a blood sample as a cell image, learn the cell image through a machine learning model to derive a learning result, and diagnose a disease in relation to the blood sample on the basis of the learning result.

2. The computer system of claim 1, comprising a lens-free shadow imaging device configured to acquire a cell image,
wherein the lens-free shadow imaging device includes:
a cell chip where the blood sample is placed;
a light-emitting diode configured to shed light on the blood sample; and
a CMOS image sensor configured to capture a lens-free shadow image of the blood sample.

3. The computer system of claim 2, further comprising a machine learning server having the machine learning model,
wherein the machine learning server is configured to receive the cell image from the lens-free shadow imaging device and learn the cell image through the machine learning model to derive a learning result.

4. The computer system of claim 3, wherein the blood sample has a plurality of blood cells, and the machine learning server is configured to cut the cell image and detect a plurality of individual cell images for each of the blood cells through an object detection algorithm and to learn the individual cell images and detect blast cells from the blood cells,
wherein the learning result includes the percentage of blast cells in blood cells in the cell image.

5. The computer system of claim 4, wherein the lens-free shadow imaging device is configured to receive the learning result from the machine learning server and diagnose a disease in relation to the blood sample on the basis of the learning result.

6. The computer system of claim 4, wherein the computer system is configured to diagnose a disease if the percentage of blast cells in the blood cells in the cell image is at or above a predetermined percentage.

7. The computer system of claim 4, wherein the blood cells are CD4+ cells.

8. The computer system of claim 1, wherein the machine learning model includes Alexnet which is based on a convolution neural network.

9. A method for a computer system, the method comprising:
acquiring a lens-free shadow image for a blood sample as a cell image;
learning the cell image through a machine learning model to derive a learning result; and
determining the percentage of blast cells in blood cells in the cell image on the basis of the learning result.

10. The method of claim 9, wherein the acquiring of a lens-free shadow image as a cell image is performed by a lens-free shadow imaging device,
wherein the lens-free shadow imaging device includes:
a cell chip where the blood sample is placed;
a light-emitting diode configured to shed light on the blood sample; and
a CMOS image sensor configured to capture a lens-free shadow image of the blood sample.

11. The method of claim 10, wherein the learning of the cell image to derive the learning result is performed by a machine learning server having the machine learning model.

12. The method of claim 11, wherein the blood sample has a plurality of blood cells, and
the learning of the cell image to derive the learning result includes:
cutting the cell image and detecting a plurality of individual cell images for each of the blood cells through an object detection algorithm; and
learning the individual cell images and detecting blast cells from the blood cells.

13. The method of claim 12, wherein the determining of the percentage of blast cells in the blood cells in the cell image is performed by the lens-free shadow imaging device.

14. A computer-readable recording medium storing an algorithm for implementing the method of claim 9 on a computer.

15. A computer-readable recording medium storing an algorithm for implementing the method of claim 10 on a computer.

16. A computer-readable recording medium storing an algorithm for implementing the method of claim 11 on a computer.

17. A computer-readable recording medium storing an algorithm for implementing the method of claim 12 on a computer.

18. A computer-readable recording medium storing an algorithm for implementing the method of claim 13 on a computer.
